(19) United States
(12) Patent Application Publication
(10) Pub. No.: US 2007/0260269 A1
(43) Pub. Date: Nov. 8, 2007

(54) Toothed vasectomy clamps and methods of using same

(76) Inventor: Joel L. Marmar, Philadelphia, PA (US)

Correspondence Address: Klehr, Harrison, Hervey,;Branzurg & Ellers LLP, 260 South Broad Street, Philadelphia, PA, 19102, US

(21) Appl. No.: 11/417,822

(22) Filed: May 4, 2006

(51) Int. Cl. A61B 17/08

(52) U.S. Cl. 606/157; 606/167

(57) ABSTRACT

Apparatus and methods for performing vasectomies are disclosed. In preferred embodiments, a clamp has a set of jaws, the distal ends of which terminate in a circular structure that is essentially a split ring. In certain embodiments, each jaw has a serrated edge that corresponds to serration on the opposite jaw, and each jaw is slightly less than a half-circle so that when closed there is a small slot or opening toward the handle side of the jaws. The jaws are adapted to grasp the vas of a patient and the slit permits a scalpel blade to cut the vas sheath longitudinally in the clamped region. Alternatively, the serrated jaws can be replaced by a single tooth on each arm of the clamp, and these teeth overlap, again leaving a single slot. Finally, in certain other embodiments, the distal ends are hooked structures and remain spaced apart even when the clamp is in the closed position.

The present invention relates to surgical instruments, and more particularly to clamps for use in vasectomy procedures

### BACKGROUND OF THE INVENTION

Male sterilization via surgery is often accomplished via a vasectomy, namely that involves removal or disruption of at least a portion of the vas deferens. Currently, vasectomies are performed by making standard scrotal incisions; however, “no-scalpel” vasectomy procedures have also been developed. No scalpel procedures are an improvement since incisions/openings into the scrotum are minimized and the attendant apprehension of the patient is reduced. The no-scalpel technic is less invasive and maybe accomplished in a shorter time than traditional vasectomy, but also presents challenges for the surgeon.

For example, the no-scalpel vasectomy and standard vasectomies requires that the vas deferens be palpated and identified before any skin opening is made. Then, the vas and surrounding skin of the scrotum is fixed securely in position with a ringed clamp, or else the vas and surrounding fascia are grasped after the skin opening is completed. As described in Marmar et al. “A Minimally Invasive Vasectomy With the No Suture, Inline Method for Vas Occlusion” (Int. J. Fertil. 46(5):257-264, 2001) is another surgical alternative. Although the treatment of the vas is quick and effective with this method, the vas and fasia must still be grasped securely to perform the “Inline Vasectomy” Regardless of the vasectomy method, a hemostat or knife pierces the scrotum, the vas deferens is grasped and secured with an encircling clamp (the type seen in FIG. 1A is preferred) and the vas is elevated above the skin level thru the skin opening. A segment of the vas is selectively secured by a skin hook, cut and then ligated/clipped and cauterized. Each step of the procedure requires different surgical tools to accommodate a relatively high degree of surgical skill and dexterity needed for this procedure. The vas deferens is an elusive structure and it must be held in place throughout the procedure since it will tend to immediately recoil into the scrotum whenever it is not secured by a clamp. The loss of the vas after the start of the procedure will require extensive dissection and manipulation often resulting in increased bleeding and swelling. Thus, any tool or device that simplifies these procedures by providing firm fixation of the vas deferens would be desirable.

Tools specifically for use in vasectomy procedures are well known, for example, U.S. Pat. No. 5,067,958—Sandhaus, discloses a device for use in a vasectomy that has non-circular, asymmetrical jaws. U.S. Pat. No. 4,920,982—Goldstein discloses a clamp having a circular jaw (FIG. 1) with an opening on the distal end that is provided so that no undue force is exerted on the scrotal skin. 3, 11. 49-59). Therefore, although this device is used in a vasectomy procedure, it is used during a different part of the procedure and for a different purpose than the invention.

None of these prior art devices, however, permits these procedures to be performed in an effective and efficient manner and with the confidence that the vas will not slip during the procedure. Therefore, there remains a long-felt yet unmet need for providing devices specifically designed to facilitate a “no scalpel”, “in line” and other vasectomies. It would further be desirable to provide such improvements in a manner that permitted their application across a variety of situations and that permitted their implementation in a cost-effective manner.

### SUMMARY OF THE INVENTION

Accordingly, it has now been found that these and other shortcomings of the prior art can be overcome by providing a clamp which has a set of jaws, the distal end of which terminate in a circular structure that is essentially a split ring. Each jaw has a serrated edge that corresponds to serration on the opposite jaw, and each jaw is slightly less than a half-circle so that when closed there is a small slit opening toward the interior side of the jaws. The circular jaws are adapted to grasp the vas of a patient and the slit permits a scalpel blade to cut the vas sheath longitudinally in the clamped region.

A number of embodiments of surgical apparatus are disclosed which have first and second members connected at a pivot point that is movable between a closed position and an open position. Each of these members has distal and proximal ends, and each of the proximal ends preferably has a handle. Each of the distal ends preferably has an at least partially arcuate portion that has a less than semi-annular section and terminates at a toothed tip. In accordance with certain aspects of the present invention, these distal ends engage one another when the apparatus is in the closed position, however, the semi-annular sections remain spaced apart from one another to form a slot so that a clamping structure is formed that less than completely encircles a body structure, which in a most preferred embodiment is a vas deferens. In certain embodiments there are a toothed edges adjacent the distal ends that are engageable with one another to form a closed joint, and this may either be a plurality of teeth or a single tooth, in which case it is preferred that the arcuate portions of the distal ends overlap each other. In preferred embodiments, the slot remaining between the arcuate portions of the distal ends is between 1.0 and 2.0 mm wide.

Alternatively, in other embodiments the surgical apparatus of the present invention comprises first and second members connected at a pivot point that is movable between a closed position and an open position. Each of these members has distal and proximal ends, and each of the proximal ends preferably has a handle. Each of the distal ends preferably has an at least partially arcuate portion that has a less than semi-annular section and terminates at a pointed tip, such that the distal end of the first member and the distal end of the second member remain spaced apart from one another when the apparatus is in the closed position and the semi-annular sections remain spaced apart from one another in the closed position to form a slot, whereby a clamping structure is formed that less than completely encircles a body structure. In such embodiments it is preferred that the first and second members includes a pointed hook portion as part of the distal ends and the hook portions of the distal ends can be either angled or straight. However, it is again preferred that the distal ends are spaced apart between 1.0 and 2.0 mm when the apparatus is in the closed position.

The present invention also relates to improved methods for performing a vasectomy that use a vas clamp comprising: first and second members pivotably connected to one another and movable between a closed position and an open position, each of the first and second members having oppositely disposed proximal and distal ends, wherein each of the distal ends includes an arcuate portion. The surgeon then moves the first and second members to the closed position around a vas deferens, thereby grasping the vas deferens so that in the closed position the first member and the second member do not completely encircle the vas deferens and leave a slot on a side of the distal ends toward the proximal end of the first and second members. The vas deferens is then pulled above skin level thru the opening in the scrotal sac and cut, completing the vasectomy. In such methods, the distal ends either preferably abut, thereby leaving a single slot on the side of the distal tips toward the proximal end of the clamp, or remain spaced apart.

### BRIEF DESCRIPTION OF THE DRAWINGS

FIG. 1 is a plan view of a multi-tooth vas clamp made in accordance with the present invention;

FIG. 2 is a side elevation view of the vas clamp shown in FIG. 1;

FIG. 3 is an enlarged elevation view of the distal end of the vas clamp shown in FIGS. 1-2;

FIG. 4 is a plan view of the distal end of the vas clamp shown in FIG. 3

FIG. 5 is a front view of the distal end of the vas clamp shown in FIG. 4;

FIG. 6 is a plan view of a single tooth vas clamp made in accordance with the present invention;

FIG. 7 is a side elevation view of the vas clamp shown in FIG. 6;

FIG. 8 is an enlarged elevation view of the distal end of the vas clamp shown in FIGS. 6-7;

FIG. 9 is a plan view of the distal end of the vas clamp shown in FIG. 8;

FIG. 10 is a front view of the distal end of the vas clamp shown in FIG. 9;

FIG. 11 is a diagrammatic view of a vasectomy procedure being conducted using the vas clamp of the present invention;

FIG. 12 is a plan view of a single tooth tenaculum vas clamp made in accordance with the present invention;

FIG. 13 is an enlarged plan view of the distal end of the vas clamp shown in FIG. 12;

FIG. 14 is a plan view of an angled single tooth tenaculum vas clamp made in accordance with the present invention;

FIG. 15 is an enlarged plan view of the distal end of the vas clamp shown in FIG. 14;

FIG. 16 is a plan view of a half-tenaculum vas clamp made in accordance with the present invention;

FIG. 17 is an enlarged plan view of the distal ends of the vas clamp shown in FIG. 16;

FIG. 18 is a plan view of the vas clamp shown in FIG. 16 in the closed position;

FIG. 19 is a plan view of an angled multi-tooth half-tenaculum vas clamp made in accordance with the present invention;

FIG. 20 is an enlarged plan view of the distal ends of the vas clamp shown in FIG. 19;

FIG. 21 is an enlarged elevation view of a first distal end of the vas clamp shown in FIG. 19;

FIG. 22 is an enlarged elevation view of a second distal end of the vas clamp shown in FIG. 19; and

FIG. 23 is a plan view of the vas clamp shown in FIG. 19 in the closed position.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

The general design and construction of the vas clamps shown herein will be familiar to those skilled in the art. The selection of materials and overall size and shape of these surgical instruments is similarly well known. Referring now to FIG. 1, a plan view of a multi-tooth vas clamp 100 made in accordance with the present invention is shown. As is typical, the clamp is comprised of a first member 102 and a second member 104 that are connected by a pivot point 106. The pivotable connection permits the clamp 100 to be opened and closed, as described in further detail below. The first member 102 and second member 104 each have a proximal end that respectively terminate in a handle portion 108,109 that is illustrated as the traditional finger loop handle, however, this structure is not part of the present invention. Similarly, the first member 102 and a second member 104 each comprise, respectively, a locking portion 120,122 that permits the clamp to be locked in a closed position, most preferably under two or more degrees of varying pressure depending upon which of two or more locking positions is chosen by the surgeon, as known in the art. As seen in the elevation view of FIG. 2, the vas clamp of FIG. 1 is substantially straight and has only a minor taper toward its distal end. In one preferred embodiment, the overall length of the vas clamp 100 illustrated in FIGS. 1-2 is about 10.5 cm.

Referring again to FIG. 1, it can be seen that the first member 102 and second member 104 terminate at their respective distal ends 110,111. In accordance with the present invention, these distal ends 110,111 each comprise a portion of a clamping structure, most preferably about one-half the clamping structure, although it is not necessary that the distal ends 110,111 be symmetrical or of equal size, as will be appreciated with reference to alternate preferred embodiments described in detail below. In the embodiment illustrated in FIG. 1, the clamping structure is substantially ring-like except for an opening on the rear, or toward the distal side of the clamping structure. In one preferred embodiment the clamping structure has an outside diameter of approximately 0.5 cm and an inside diameter of approximately 0.4 cm. As explained in further detail below, the provision of this opening permits improvements in the vasectomy procedure.

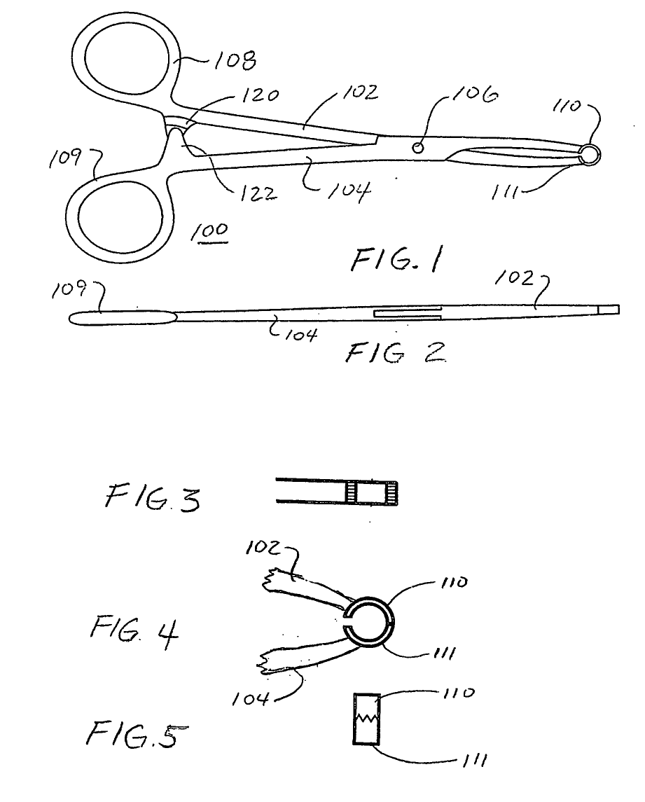

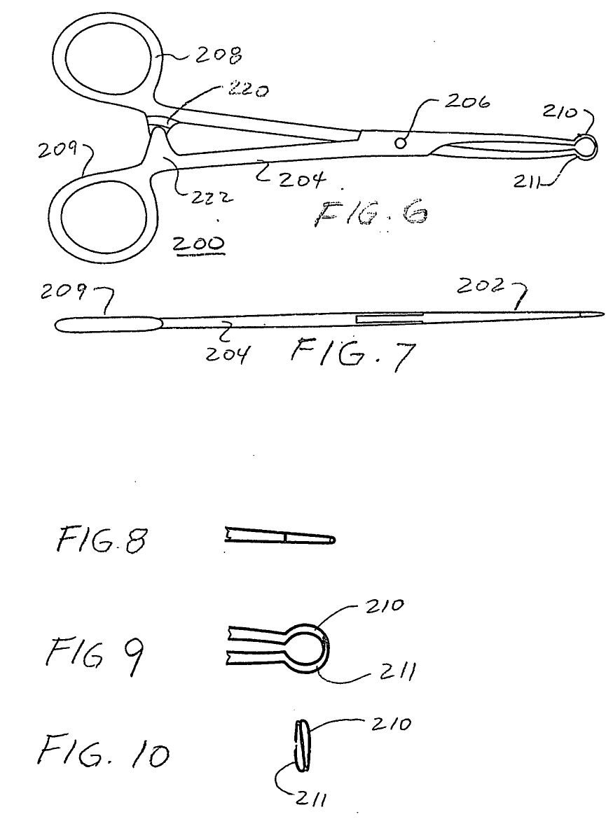

Further details of the vas clamp illustrated in FIG. 1 are visible in FIGS. 3-5. FIG. 3 is an enlarged side elevation view of the distal ends 110,111 of the vas clamp 100 shown in FIG. 1 and illustrates the serrations or teeth provided in this particular embodiment. In one preferred embodiment the thickness of the distal ends 110,111 is between about 1.75-2.0 mm and comprises three teeth. The engagement of these teeth is illustrated in FIG. 5. Referring now to FIG. 4, a plan view of the distal end 110,111 of the vas clamp shown in FIG. 3 is shown along with a broken away portion of the first member 102 and second member 104. As seen in FIG. 4, the opening between the distal ends 110,111 discussed above with reference to FIG. 1 is clearly seen. In one preferred embodiment, this opening is about 1.0 mm across.

Another preferred embodiment of the present invention is illustrated in FIG. 6, which is similar to FIG. 1 in that it is a plan view of a vas clamp 200 made in accordance with the present invention, however, in this embodiment the distal end of the clamp 200 has a single tooth. As described above, the embodiment of FIG. 6 also comprises first and second members 202,204 connected by a pivot 206 and terminating at their respective proximal ends with handle portions 208,209 and including locking structures 120,122. As seen in FIG. 7, unlike the embodiment described above, the first and second members 202,204 taper as they approach the distal ends 210,211.

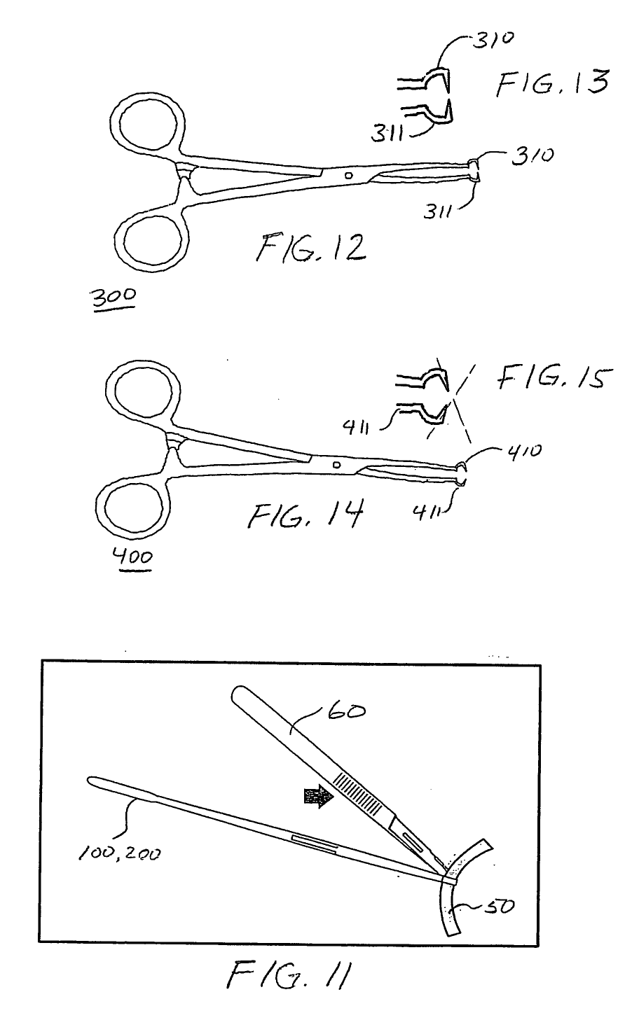

Further details of the single tooth vas clamp illustrated in FIG. 6 are visible in FIGS. 8-10. FIG. 8 is an enlarged side elevation view of the distal ends 210,211 of the vas clamp 200 shown in FIG. 6 and illustrates the single tooth provided in this particular embodiment. In one preferred embodiment the thickness of the distal ends 210,211 is about 1.0 mm. The engagement of these teeth is illustrated in FIG. 10, as illustrated; the curved, arcuate teeth are tapered and overlap. In one preferred embodiment, the overall thickness of this overlapped structure is about 1.5 mm. Referring now to FIG. 9, a plan view of the distal ends 210,211 shown in FIG. 8 is shown along with a broken away portion of the first member 202 and second member 204. As seen in FIG. 9, the opening between the distal ends 110,111 discussed above with reference to FIG. 1 is clearly seen. In one preferred embodiment, this opening is about 1 mm (0.1 cm) across.

FIG. 11 is a diagrammatic view of a vasectomy procedure being conducted using the vas clamp of the present invention. As explained above, an important part of a “no scalpel”, “in line” or standard vasectomy is the ability to firmly grasp the vas, and not have to continually manipulate this duct, thereby minimizing dissection, swelling and reducing the time of the procedure. In FIG. 11, the procedure is to a point where the vas 50 has been grasped by a vas clamp 100,200 made in accordance with the present invention. The curved, arcuate distal ends enable a firm grasp without crushing the vas 50 while the opening described above on the proximal side is large enough to permit incising the vas using a scalpel 60, most preferably carrying a #15 blade. As a result of both the firm grasp and the ability to make a clean longitudinal incision provided by the slotted opening, the vasectomy procedure can be readily completed with less time, trauma and chance of error.

Additional embodiments of the present invention are disclosed in FIGS. 12-15. Referring now to FIG. 12, a plan view of a single tooth tenaculum vas clamp 300 made in accordance with the present invention is shown. This clamp 300 is made substantially in accordance with the structures described above with reference to FIGS. 1-10. However, as see in FIG. 12, the distal ends 310,311 may or may not touch to form a closed grasping structure, as described above. Moreover, the distal ends 310,311 of this embodiment are have pointed ends hooked so they perform a hooking function during the procedure. Further details of the distal ends 310,311 are shown in FIG. 13, which is an enlarged plan view of the distal end of the vas clamp 300 shown in FIG. 12. In a preferred embodiment, the respective arcuate sections are about 2.5 mm across and from the tip of the hook to the bottom of the curve is about 1.5 mm on each side, thus leaving a 2.0 mm gap.

A variation of this device is shown in FIG. 14 is a plan view of an angled single tooth tenaculum vas clamp 400 made in accordance with the present invention. Unlike the vas clamp 300 shown in FIGS. 12-13, this embodiment has tips that are angled, in other words, the axes of the most distal potion of the distal ends 410,411 intersect, as shown by the dashed lines in the enlarged plan view of the distal end of the vas clamp 400 shown in FIG. 15.

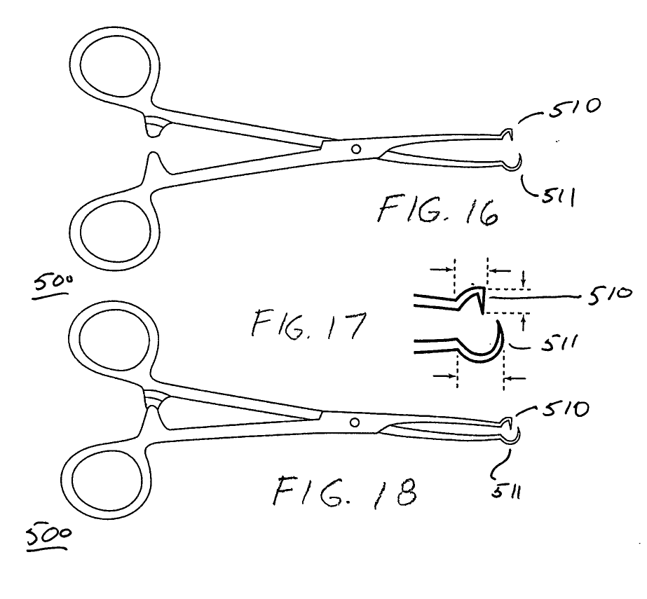

A different embodiment of the present invention is disclosed in FIGS. 16-18. Referring now to FIG. 16, a plan view of a half-tenaculum vas clamp 500 made in accordance with the present invention is shown. This clamp 500 is made substantially in accordance with the structures described above with reference to FIGS. 1-15. However, as see in FIG. 16, the distal ends 510,511 do not touch to form a closed grasping structure, as described above. However, unlike the other preferred embodiments, one distal end 510 has a different shape than the other distal end 511. As described immediately above, the distal ends 510,511 of this embodiment have pointed ends that are shaped so they perform a hooking function during the procedure; however, they are not symmetrical. Further details of the distal ends 510,511 are shown in FIG. 17, which is an enlarged plan view of the distal end of the vas clamp 500 shown in FIG. 12. In a preferred embodiment, one of the respective arcuate sections 510 is about 2.5 mm across and from the tip of the hook to the bottom of the curve is about 1.5 mm. The other distal end 411 is a fuller “hook” preferably having a width of about 5 mm. As seen in FIG. 18, when this pair of distal ends 510,511 is closed, the pointed tip of the first distal end 510 is located at about the center of the arc described by the second distal end 511.

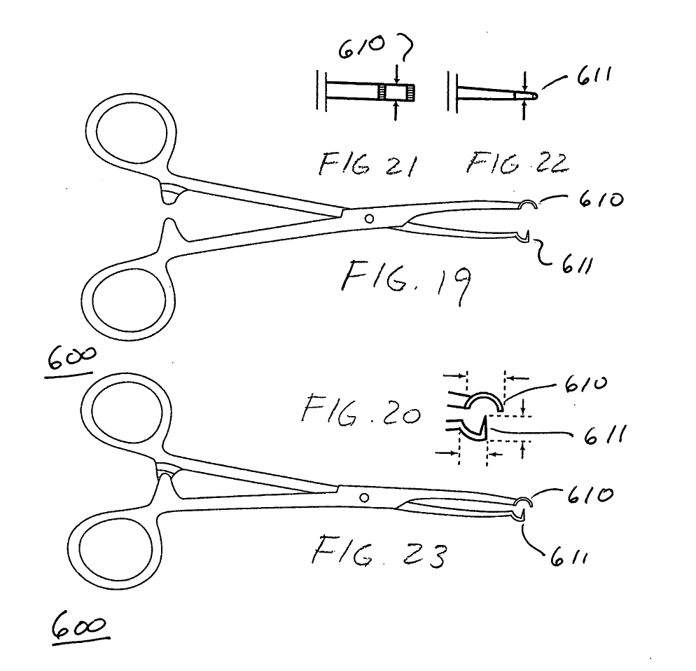

A further alternate embodiment of the present invention is disclosed in FIGS. 19-23. Referring now to FIG. 19, a plan view of a multi-tooth half-tenaculum vas clamp 600 made in accordance with the present invention is shown. This clamp 600 is made substantially in accordance with the structures described above with reference to FIGS. 1-18. As seen in FIG. 19 and in the enlarged plan view of the distal ends 610,611 of the vas clamp 600 shown in FIG. 20, as described above with reference to FIGS. 16-18, the distal ends 610,611 are not symmetric. A first distal end 610 is an arcuate section with a width of about 5 mm, while the second distal end 611 is a half tenaculum, as described above. Thus as illustrated in FIG. 23, as with the embodiment described with reference to FIG. 18, the distal ends of the jaws 610,611 align in the closed position so that the point of the tenaculum that terminates the second distal end 611 is approximately centered over the arcuate distal end 610. As best illustrated in the elevation views of FIGS. 21-22, the distal ends 610,611 further differ from the embodiments discussed immediately above in that the arcuate first distal end 610 has a multi-tooth jaw.

Although certain embodiments of the present invention have been described with particularity, these embodiments are illustrative and do not limit the present invention. In particular, the present invention is not limited to a particular surgical procedure or size of device, nor to the type or vessel, duct, tube, organ or body structure that can be manipulated. Upon review of the foregoing, numerous adaptations, modifications, and alterations will occur to those skilled in the art. These will all be, however, within the spirit of the present invention. Accordingly, reference should be made to the appended claims in order to ascertain the true scope of the present invention

What is claimed is:

1. Surgical apparatus comprising: first and second members connected at a pivot point and movable between a closed position and an open position, each of said first and second members having distal and proximal ends, each of said proximal ends including a handle portion, and each of said distal ends including an at least partially arcuate portion that has a less than semi-annular section and terminates at a toothed tip, wherein said distal end of said first member and said distal end of said second member engage one another when the apparatus is in the closed position and said semi-annular sections remain spaced apart from one another in said closed position to form a slot, whereby a clamping structure is formed that less than completely encircles a body structure.

2. The apparatus of claim 1 wherein each of said first and second members includes a toothed edge adjacent said distal ends, said toothed edges being engageable with one another to form a closed joint.

3. The apparatus of claim 1 wherein each of said toothed edges of said first and second members comprise a plurality of teeth.

4. The apparatus of claim 1 wherein of each of said toothed edges of said first and second members comprise a single tooth.

5. The apparatus of claim 4 wherein said arcuate portions of said distal ends overlap each other.

6. The apparatus of claim 1 wherein said slot is between 1.0 and 2.0 mm wide.

7. The apparatus of claim 1 wherein said first and second members are tapered.

8. The apparatus of claim 1 wherein said surgical apparatus is a vas clamp.

9. Surgical apparatus comprising: first and second members connected at a pivot point and movable between a closed position and an open position, each of said first and second members having distal and proximal ends, each of said proximal ends including a handle portion, and each of said distal ends including an at least partially arcuate portion that has a less than semi-annular section and terminates at a pointed tip, wherein said distal end of said first member and said distal end of said second member remain spaced apart from one another when the apparatus is in the closed position and said semi-annular sections remain spaced apart from one another in said closed position to form a slot, whereby a clamping structure is formed that less than completely encircles a body structure.

10. The apparatus of claim 9 wherein each of said first and second members includes a pointed hook portion as part of said distal ends.

11. The apparatus of claim 10 wherein said hook portions of said distal ends are angled.

12. The apparatus of claim 9 wherein said distal ends are spaced apart between 1.0 and 2.0 mm when the apparatus is in the closed position.

13. The apparatus of claim 9 wherein said first and second members are tapered.

14. The apparatus of claim 9 wherein said surgical apparatus comprises a first arcuate distal end and a second hook-shaped distal end.

15. The apparatus of claim 9 wherein said surgical first and second distal ends are not symmetrical.

16. The apparatus of claim 14 wherein the first arcuate distal end further comprises serrated teeth.

17. The apparatus of claim 9 wherein said surgical apparatus is a vas clamp.

18. A method for performing a vasectomy, said method comprising the steps of:
a) providing a vas clamp apparatus comprising: first and second members pivotably connected to one another and movable between a closed position and an open position, each of the first and second members having oppositely disposed proximal and distal ends, wherein each of the distal ends includes an arcuate portion;
b) moving the first and second members to the closed position around a vas deferens thereby grasping the vas deferens so that in the closed position the first member and the second member do not completely encircle the vas deferens and leave a slot on a side of the distal ends toward the proximal end of the first and second members;
c) pulling the vas deferens away from a scrotal sac; and
d) cutting the vas deferens.

19. The method according to claim 15 wherein the distal ends abut, leaving a single slot.
